(19) United States
(12) Patent Application Publication
(10) Pub. No.: US 2003/0118606 A1
(43) Pub. Date: Jun. 26, 2003

(54) Anticancer compositions

(76) Inventor: Akikuni Yagita, Tokyo (JP)

Correspondence Address: Luke A Kilyk, Kilyk & Bowersox, 53 A Lee Street, Warrenton, VA, 20186, US

(21) Appl. No.: 10/221,333

(22) PCT Filed: Mar. 23, 2001
(86) PCT No.: PCT/JP01/02315

(30) Foreign Application Priority Data
Mar. 24, 2000 (JP) 2000-128616

(51) Int. Cl. A61K 35/84

(52) U.S. Cl. 424/195.15

(57) ABSTRACT

There is provided a composition or a composition for oral administration which contains a mycelium fraction of a product obtained by incubation of mycelia of Ganoderma boninense as a main ingredient and induces the production of interleukin 12 (IL-12) or a supplementary food preparation for health by oral administration which is taken with an expectation of anticancer effect. There are also provided a commercial medium carrying the information concerning the above and a commercial method using the said commercial medium.

### TECHNICAL FIELD

The present invention relates to a composition or a composition for oral administration paying attention for induction of the production of interleukin 12 (IL-12) or to a supplementary food preparation for health which is taken with an expectation of anticancer effect paying attention for induction of the production of IL-12.

### BACKGROUND OF THE INVENTION

In the selection of substances useful for the prevention and therapy of malignant neoplasm or cancer, its direct action to cancer cells has been considered to be important. Although immunopotentiators have been noted to be useful for the therapy of cancer, anticancer effect of all compounds which are obtained as immunopotentiators is weak and a sufficient therapeutic effect for cancer has not been achieved by the immunotherapy only or even by the joint therapy with chemotherapy.

Dr. Yagita who is the present inventor has previously paid his attention to the usefulness of a substance which induces IL-12 in vivo as an epoch-making means in the therapy of cancer and found AHCC which is a processed product of mycelia of Cortinellus shiitake has such a function. Although it has been known already that IL-12 has an anticancer effect, side effect is resulted when IL-12 per se is directly administered into organism whereby the patient is not endurable the therapy and, therefore, IL-12 per se has not been able to be used as an anticancer agent. However, a preparation containing AHCC reported by Yagita achieved a significant therapeutic and life-prolonging effect in the therapy of cancer. Thus, by administration of an effective dose of AHCC by which IL-12 is able to be induced in vivo, Yagita has achieved the therapeutic object for cancer (Japanese Patent Laid-Open No. 10/139,670).

IL-12 has a potentiating action for the production of interferon γ (IFN γ) and an activating and potentiating action for natural killer (NK) cells, LAK cells (lymphokine activated killer cells) and killer T cells having a role of cell-mediated immunity in vivo. IFN γ is cytokine which induces the immune response of organism to such a state that T helper-1 cell (Th1) acts. The state where Th1 acts means a state where natural killer-T (NKT) cells and killer T cells are apt to achieve their effect or, in other words, a state where IL-2 and IL-12 are abundantly produced. Killer T cells and LAK cells have been known as the cells participating in immune of cancer. With regard to NK cells, it has been also reported that they participate in anticancer action in vivo but it has been proved by Yagita that, in the NK cells, clinical anticancer effect does not correlate to the activity but, rather, the induced production amount of IL-12 shows an entirely reversely correlation to the NK activity. Thus, it may be concluded that NK cells do not participate in the anticancer action in human being.

At present, it has been established by Yagita that a substance having an ability of inducing the production of IL-12 has a possibility of becoming a prominent anticancer substance and that has been ascertained in the present invention as well.

However, in some patients suffering from cancer, production of IL-12 is not well induced even by administration of AHCC resulting in no therapeutic effect and, even if production of IL-12 is induced, the therapeutic effect is not achieved. Under such circumstances, there has been a further demand for a new agent for the therapy of cancer acting by a different mechanism from the anticancer action of AHCC.

### DISCLOSURE OF THE INVENTION

The present inventor has found a composition derived from mycelia of a kind of mushroom having a novel inducing ability for IL-12 production which is different from the IL-12 inducing effect by AHCC and being effective to patients suffering from progressive cancer or terminal cancer or where immune response of a T helper 2 cell (Th2) system is mostly functioning whereupon the present invention has been accomplished.

Thus, an embodiment of the present invention is a composition which induces the production of IL-12 containing a mycelium fraction of the product obtained by incubation of mycelia of Ganoderma boninense as a main ingredient.

Another embodiment of the present invention is a composition which induces the production of IL-12 containing a mycelium fraction of the product obtained by liquid incubation of mycelia of Ganoderma boninense as a main ingredient.

Another embodiment of the present invention is a composition for oral administration which induces the production of IL-12 containing a mycelium fraction of the product obtained by incubation of mycelia of Ganoderma boninense as a main ingredient.

Another embodiment of the present invention is a composition for oral administration which induces the production of IL-12 containing a mycelium fraction of the product obtained by liquid incubation of mycelia of Ganoderma boninense as a main ingredient.

Another embodiment of the present invention is a composition for oral administration which induces the production of IL-12 containing a mycelium fraction of the product obtained by incubation of mycelia of Ganoderma boninense as a main ingredient which is characterized in that, as the dose achieving the inducing ability for the production of IL-12 in vivo, the main ingredient is administered per os in a dose of 100 mg to 2,000 mg/kg body weight/day.

Another embodiment of the present invention is a composition for oral administration which induces the production of IL-12 containing a mycelium fraction of the product obtained by liquid incubation of mycelia of Ganoderma boninense as a main ingredient which is characterized in that, as the dose achieving the inducing ability for the production of IL-12 in vivo, the main ingredient is administered per os in a dose of 100 mg to 2,000 mg/kg body weight/day.

Another embodiment of the present invention is a composition which induces the production of IL-12 containing a mycelium fraction of the product obtained by incubation of mycelia of Ganoderma boninense as a main ingredient which is characterized in that it is useful for the patient suffering from progress cancer or terminal cancer.

Another embodiment of the present invention is a composition which induces the production of IL-12 containing a mycelium fraction of the product obtained by liquid incubation of mycelia of Ganoderma boninense as a main ingredient which is characterized in that it is useful for the patient suffering from progress cancer or terminal cancer.

Another embodiment of the present invention is a composition for oral administration which induces the production of IL-12 containing a mycelium fraction of the product obtained by incubation of mycelia of Ganoderma boninense as a main ingredient which is characterized in that it is useful for the patient suffering from progress cancer or terminal cancer.

Another embodiment of the present invention is a composition for oral administration which induces the production of IL-12 containing a mycelium fraction of the product obtained by liquid incubation of mycelia of Ganoderma boninense as a main ingredient which is characterized in that it is useful for the patient suffering from progress cancer or terminal cancer.

Another embodiment of the present invention is a composition for oral administration which induces the production of IL-12 containing a mycelium fraction of the product obtained by incubation of mycelia of Ganoderma boninense as a main ingredient and is effective to patients suffering from progressive cancer or terminal cancer which is characterized in that, as the dose achieving the inducing ability for the production of IL-12 in vivo, the main ingredient is administered per os in a dose of 100 mg to 2,000 mg/kg body weight/day.

Another embodiment of the present invention is a composition for oral administration which induces the production of IL-12 containing a mycelium fraction of the product obtained by liquid incubation of mycelia of Ganoderma boninense as a main ingredient and is effective to patients suffering from progressive cancer or terminal cancer which is characterized in that, as the dose achieving the inducing ability for the production of IL-12 in vivo, the main ingredient is administered per os in a dose of 100 mg to 2,000 mg/kg body weight/day.

Another embodiment of the present invention is a composition which induces the production of IL-12 containing a mycelium fraction of the product obtained by incubation of mycelia of Ganoderma boninense as a main ingredient which is characterized in that it is useful for patients suffering from cancer where a Th2 cell system immune response is mainly functioning.

Another embodiment of the present invention is a composition which induces the production of IL-12 containing a mycelium fraction of the product obtained by liquid incubation of mycelia of Ganoderma boninense as a main ingredient which is characterized in that it is useful for patients suffering from cancer where a Th2 cell system immune response is mainly functioning.

Another embodiment of the present invention is a composition for oral administration which induces the production of IL-12 containing a mycelium fraction of the product obtained by incubation of mycelia of Ganoderma boninense as a main ingredient which is characterized in that it is useful for patients where a Th2 cell system immune response is mainly functioning.

Another embodiment of the present invention is a composition for oral administration which induces the production of IL-12 containing a mycelium fraction of the product obtained by liquid incubation of mycelia of Ganoderma boninense as a main ingredient which is characterized in that it is useful for patients where a Th2 cell system immune response is mainly functioning.

Another embodiment of the present invention is a composition for oral administration which induces the production of IL-12 containing a mycelium fraction of the product obtained by incubation of mycelia of Ganoderma boninense as a main ingredient and is effective to patients suffering from cancer where a Th2 cell system immune response is mainly functioning which is characterized in that, as the dose achieving the inducing ability for the production of IL-12 in vivo, the main ingredient is administered per os in a dose of 100 mg to 2,000 mg/kg body weight/day.

Another embodiment of the present invention is a composition for oral administration which induces the production of IL-12 containing a mycelium fraction of the product obtained by liquid incubation of mycelia of Ganoderma boninense as a main ingredient and is effective to patients suffering from cancer where a Th2 cell system immune response is mainly functioning which is characterized in that, as the dose achieving the inducing ability for the production of IL-12 in vivo, the main ingredient is administered per os in a dose of 100 mg to 2,000 mg/kg body weight/day.

Another embodiment of the present invention is any of the above-mentioned compositions, characterized in that, the said composition is used together with a composition where a saccharide having an α-1→3 stereostructure as a main ingredient.

Another embodiment of the present invention is any of the above-mentioned compositions for oral administration, characterized in that, the said composition is used together with a composition where a saccharide having an α-1→3 stereostructure as a main ingredient.

Another embodiment of the present invention is any of the above-mentioned compositions for oral administration where the said composition is a supplementary food preparation for health by oral administration.

Another embodiment of the present invention is a commercial medium carrying the content concerning the above-mentioned present invention.

Another embodiment of the present invention is a commercial method utilizing the content concerning the above-mentioned present invention.

### BRIEF DESCRIPTION OF THE DRAWINGS

FIG. 1 is a drawing which shows a protocol of the experiment.

FIG. 2 is a drawing which shows IL-12 (pg/ml) in serum in cancer-bearing mice.

### BEST MODE FOR CARRYING OUT THE INVENTION

The present invention will now be illustrated in more detail as hereunder and the following detailed description is just exemplary and for illustration only and does not limit the present invention at all.

Technical and scientific terms used in the present specification have the meanings which are normally understood by the persons having the common knowledge in the technical field to which the present invention belongs unless otherwise defined. In the present specification, cited references such as publications are introduced in such a sense that whole of them are entirely described in the present specification by way of citing them.

As an inducer for the production of IL-12, the present inventor has found that, in addition to substances such as AHCC which effectively induces the production of IL-12 particularly in the patients suffering from primary cancer, there are substances such as a mycelium fraction of Ganoderma boninense according to the present invention which characteristically achieve an inducing effect for the production of IL-12 even for patients suffering from progressive cancer or terminal cancer. It has been further found that AHCC induces the production of IL-12 in patients and mice suffering from cancer where immune response of a Th1 system is mainly functioning while the above-mentioned mycelium fraction of Ganoderma boninense induces IL-12 in patients and mice suffering from cancer where immune response of a Th2 system is mainly functioning.

Here, the term reading patients and mice suffering from cancer where immune response of a Th1 system is functioning means that,

(A) in terms of genetic level,

(1) in the case of mice, it stands for mice which are apt to produce TNFα, IFNγ or IL-12 such as B10 mice or B10A mice in the case of congenic mice and

(2) in the case of human being, it stands for human being who is apt to produce TNFα, IFNγ or IL-12 or, to be more specific, human being having human leukocyte antigen (HLA) of a type of HLA-B51, B61, B62, B8, B27, B52 or the like; and

(B) in terms of pathology, it stands for patients suffering from cancer in a state of primary cancer or in a state of a little progressive cancer where immunological competence is not so much suppressed or for the case where, even in the case of progressive cancer, tumor was able to be excised and stands for the patients suffering from cancer in a state where TNFα, IFNγ or IL-12 is apt to be produced or, to be more specific, in a state where immunosuppressive acidic protein (IAP) is not more than 600 μg/ml and IL-10 is not more than 600 pg/ml.

Then, the term reading patients and mice suffering from cancer where immune response of a Th2 system is functioning means that,

(A) in terms of genetic level,

(1) in the case of mice, it stands for mice which hardly produce TNFα, IFNγ or IL-12 such as Balb/c mice or B10D₂ mice in the case of congenic mice and

(2) in the case of human being, it stands for human being who hardly produces TNFα, IFNγ or IL-12 or, to be more specific, human being having human leukocyte antigen (HLA) of a type of HLA-B35, B7, B39 or the like; and

(B) in terms of pathology, it stands for patients suffering from progressive terminal cancer where TNFα, IFNγ or IL-12 is hardly produced or, to be more specific, for patients suffering from cancer where IAP is 600 μg/ml or more and IL-10 is 600 pg/ml or more.

An embodiment of the present invention is a composition where attention is paid on induction for the production of interleukin (IL-12) or, particularly, a composition which is able to induce IL-12 even for the patients where production amount of IL-12 is insufficient even when an IL-12 production inducer comprising the known mushroom components such as AHCC (K. K. Amino Up) is administered.

Still another embodiment of the present invention may be that the above-mentioned composition is a composition for oral administration which is characterized in being administered per os.

Further embodiment of the present invention is a supplementary food preparation for health by oral administration which is taken with an expectation of anticancer effect using an interleukin 12 (IL-12) production inducing ability as an index or, more particularly, it is a supplementary food preparation for health by oral administration which is taken with an expectation of anticancer effect containing a substance having an interleukin 12 (IL-12) production inducing ability and is applied to the patients where production amount of IL-12 is insufficient even when an IL-12 production inducer comprising the known mushroom components such as AHCC (K. K. Amino Up) is administered.

The composition, the composition for oral administration or the supplementary food preparation for health by oral administration in accordance with the present invention is effective for the therapy of lung cancer (lung squamous carcinoma, lung adenocarcinoma and small-cell lung cancer), thymoma, thyroid cancer, prostatic cancer, renal cancer, bladder cancer, colon cancer, rectum cancer, esophageal cancer, cecum cancer, urinary tract cancer, breast cancer, uterine cervex cancer, brain tumor, cancer of the tongue, pharyngeal cancer, nasal cavity cancer, laryngeal cancer, stomach cancer, hepatic cancer, bile duct cancer, testicular cancer, ovarian cancer, cancer of uterine body, metastatic bone cancer, malignant melanoma, osteosarcoma, malignant lymphoma, plasmacytoma, liposarcoma and the like although the present invention is not limited those cancers. It is preferably administered particularly to patients where IL-12 value is low (such as that not more than 7.8 pg/ml) even when an IL-production inducer such as AHCC (K. K. Amino Up) is administered.

With regard to measurement of the amount of the induced IL-12, there is induction of IL-12 in a sufficient amount in serum according to the experimental example using mice which will be mentioned later and, even when a indirect measurement as in human being is not conducted, the measurement is possible using a measuring kit by an enzyme-linked immunosorbent assay (ELISA). In this experimental system using mice, a substance which induces the production of IL-12 is continuously administered per os and its inducing ability for IL-12 production can be tested by an increase in the amount of IL-12 in blood thereafter.

In human being, it is not possible to directly measure the amount of IL-12 in blood due to the presence of an inhibitor in blood and, for example, measurement of the amount of IL-12 induced in the patient suffering from cancer is carried out using a culture liquid which is prepared by incubation of peripheral blood mono nuclear cell fraction separated and prepared from blood of the said patient suffering from cancer together with a stimulant followed by removing the cells by means of centrifugal precipitation. Separation of mono nuclear cell fraction from blood may be carried out by a known method and, for example, it may be easily carried out by a specific gravity centrifugal method using a Ficoll-Conray liquid or the like. Numbers of the cells used for the incubation are 0.5×10⁶ cells/ml to 1×10⁷ cells/ml and, preferably, 1×10⁶ cells/ml. With regard to a substance which stimulates the cells, phytohemagglutinin (PHA) which has been used as a mitogen is added so as to make its final concentration 0.1˜100 μg/ml or, preferably, 1˜20 μg/ml and then incubation is carried out. The substance which stimulates the cells is not limited to PHA but any substance may be used so far as it stimulates the cells and is able to produce an immunophysiologically active substance for achieving the object of the present invention and its examples are PMA (phorbol 12-myristate-13-acetate), PMA+ionomycin, LPS (lipopolysaccharide), PWM (poke weed mitogen), etc. In measuring the amount of IL-12, clinical and biochemical tests which have been known per se may be utilized and a measuring kit using an enzyme-linked immunosorbent assay (ELISA) available from R&D Systems or from MBL may be used. Here, the term of inducing ability for the production of IL-12 means a function where the amount of IL-12 produced when peripheral blood mono nuclear cell fraction is stimulated is potentiated to an extent of not more than 7.8 pg/ml or a function where the amount of IL-12 produced is potentiated before administration of some substance.

A composition which is an embodiment of the present invention contains a mycelium fraction of Ganoderma boninense as an active ingredient having an ability of inducing the production of IL-12.

A composition which is an embodiment of the present invention containing a mycelium fraction of Ganoderma boninense as an active ingredient having an ability of inducing the production of IL-12 has a big difference from the known AHCC in an inducing ability for the production of IL-12 in each progressive stage of cancer. The composition of the present invention where a mycelium fraction of Ganoderma boninense is an effective ingredient shows a sufficient inducing ability for the production of IL-12 even in the early stage of cancer and, to be characteristic, even in the progressed terminal cancer, it achieves the same or even stronger inducing ability for the production of IL-12. On the other hand, although AHCC achieves a characteristic inducing ability for the production of IL-12 in the early stage of cancer, its inducing ability decreases as the cancer proceeds.

A composition which is an embodiment of the present invention containing a mycelium fraction of Ganoderma boninense as an active ingredient having an ability of inducing the production of IL-12 has a big difference from the known AHCC in an inducing ability for the production of IL-12 even in patients suffering from cancer where the immune response mainly functioning in organism is an immune response of a Th2 system.

Dose of the composition in accordance with the present invention is about 1˜2,000 mg/kg body weight per day or, more preferably, about 100˜2,000 mg/kg body weight per day while the term for administration is from about ten days to about one year and the frequency of administration is from once to several tens times per month. Preferably, it is administered per os. It goes without saying that administering dose is decreased and the compound is prepared into a quality which is endurable for parenteral administration whereby parenteral administration (including injection by, for example, subcutaneous, intramuscular, intravenous and intracutaneous routes) is possible.

The above-mentioned mycelium fraction of Ganoderma boninense according to the present invention has been known as a food material. It is disclosed, for example, in Japanese Patent Publication No. 63/61,910. The above-mentioned mycelium fraction of Ganoderma boninense is manufactured by subjecting the mycelia obtained by incubation of incubated mother cells of Ganoderma boninense to, for example, a liquid incubation in a medium containing appropriate nutrient sources followed by recovering therefrom. It is also possible that mycelia are collected from the culture liquid, the said mycelia are extracted with a solvent or, preferably, with hot water, the resulting extract is sterilized by a membrane filter and the filtrate is concentrated and dried to give a powdery product. Those are defined as a mycelium fraction of a product obtained by incubation of mycelia of Ganoderma boninense. Incidentally, as shown in Examples, a solid fraction obtained from a culture liquid is directly washed and dried as a mycelium fraction and is used in the present invention.

Oral preparations are made into tablets, diluted powder, capsules, syrup, etc. In making into the preparations, it goes without saying that necessary additives such as diluting agent, disintegrating agent, binder and/or lubricant, etc. which have been known may be added and the conventional means is used. If necessary, it is also possible to further add corrigent, colorant, flavor, stabilizer, bactericide and/or antiseptic agent, etc.

Parenteral preparations are made into aqueous or non-aqueous sterilized injection solution which contains antioxidant, buffer and/or bacterium-controlling agent and may contain a solute making isotonic to the blood of a patient or into aqueous or non-aqueous sterilized suspension which may contains suspending agent or thickening agent. The amount of the formulation for one administration or two or more administrations may be provided by placing, for example, in a tightly sealed ampoule or vial. It is also possible to preserve in a freeze-dried state which only requires addition of a sterilized liquid carrier immediately before use.

The above-mentioned composition inducing the production of IL-12 which is an embodiment of the present invention may be a composition for oral administration containing an effective amount of a mycelium fraction of a product obtained by incubation of mycelia of Ganoderma boninense and is able to be administered per os.

A composition for oral administration containing an effective amount of a mycelium fraction of the product being obtained by incubation of mycelia of Ganoderma boninense which is an embodiment of the present invention may be a supplemental food preparation for health by oral administration which can be expected to have an anticancer effect as a result of its administration.

As hereunder, the composition, the composition for oral administration and the supplemental food preparation for health by oral administration in accordance with the present invention may be in some cases just referred to as ILY.

The above-mentioned ILY makes the novel immunotherapy for cancer (NITC) which is practiced by the present inventor more effective. Outline of the novel immunotherapy for cancer practiced by Dr. Hirokuni Yagita who is the present inventor is as follows. Thus, its main points are an immunological cancer therapy using a biological response modulating preparation (BRM preparation) where induction of IL-12, activation of NKT cells and inhibition of neovascularization are markers. 1) With regard to a neovascularization inhibitor, Better Shark MC or LO (K. K. SeishinKigyo) which is a preparation of cartilage of shark (Japanese Patent No. 3,103,513) is used in a dose of 20 g per day. 2) With regard to a method for the induction of IL-12, a) ILX (Yugen Kaisha Tozai Iyaku Kenkyusho) is used in a dose of 3˜20 g per day or, preferably, 6.0 g per day (or 6.0 g per day of AHCC; its use has been ceased at present) for patients where immune response of a Th1 system is mainly functioning and b) PSK (Sankyo) is used in the dose of 1˜20 g per day or every other day or, preferably, 3.0 g per day or every other day for patients where immune response of a Th2 system is mainly functioning. 3) With regard to a method for the activation of NKT cells, a composition where a saccharide having an α1→3 stereostructure which is a substance for stimulating an NK cell antigen receptor (NKR-P1) as a main ingredient is used. Dose of the composition where a saccharide having an α1→3 stereostructure which is a substance for activating the NKT cells by selectively acting the NKR-P1 (natural killer P1) of those NKT cells as a main ingredient is about 1˜2,000 mg/kg body weight and it is preferably administered per os for from ten days to 12 months at the rate of once to 31 times per month. With regard to a saccharide substance having an α1→3 stereostructure, at least one which is selected from the followings is an active ingredient. They are nigero-oligosaccharide (a saccharide having 3-O-α-D-glucopyranosyl-D-glucose as a constituting unit) (examples of the commercial products are: Nigero-origosaccharide Liquid Saccharide; sold by Takeda Shokuhin Kogyo K. K.; main nigero-oligosaccharides contained therein are \mathring{1} nigerose: α-D-Glc p-(1→3)-D-Glc, \mathring{2} nigerosyl glucose: α-D-Glc p-(1→3)-α-D-Glc p-(1→4)-α-D-Glc and \mathring{3} nigerosyl maltose: α-D-Glc p-(1→3)-α-D-Glc p-(1→4)-D-Glc p-(1→4)-D-Glc; where Glc is glucose and p is pyranose), fucoidan (examples are fucoidan derived from mozuku (a seaweed of the family Spermatochnaceae) produced in Okinawa, F-fucoidan/sulfated fucan derived from Kjellmaniae crassifolia: a saccharide solely comprising fucose, G-fucoidan/sulfated fucogalactan: a saccharide comprising galactose and fucose, U-fucoidan derived from Kjellmaniae crassifolia: a saccharide comprising glucoronic acid, mannose and fucose) (fucoidan is a polysaccharide containing sulfated fucose and is prepared, for example, by such a manner that sea tangle is ground and made into chips, water-soluble components are extracted, the residue after extraction is removed by centrifugal separation and low-molecular substances such as iodine and sodium chloride are removed by ultrafiltration followed by freeze-drying; sold by Takara Shuzo), oligosaccharide sulfate (such as that sold by K. K. Shirako; an extract derived from susabi nori (a kind of seaweed) and its main components are an oligosaccharide of galactan sulfate of α1→3 bond and an oligosaccharide of galactan sulfate comprising α1→3 bond and α1→4 bond), etc. Incidentally, the compound is not limited to those but substances which are saccharide substances of an α1→3 stereostructure (saccharide component having an α1→3 glucoside bond structure) and have an activating ability for NKT cells by selectively acting the NKR-P1 (natural killer P1) of NKT cells are widely covered. The substance having an activating ability for NKT cells may be a composition containing polysaccharide having the said structure and/or 2˜10 oligosaccharides.

With regard to a neovascularization inhibitor which is the first main point in the above-mentioned novel immunotherapy, cartilage of shark which is best in terms of efficacy, safety, easiness, economy, etc. was selected and improved to develop Better Shark MC or LO as a result of various basic investigations. At present, it is administered per os when the patient is hungry in a dose of 20 g per day by dividing into two or three times.

With regard to an IL-12 inducer which is the second main point, ILX (formerly, AHCC) which acts mouse or human being where an immune response of a Th1 system is mainly functioning is administered per os in a dose of, for example, about 3 g/day for the cases of early cancer and the cases after operation and in a dose of, for example, about 6 g/day for the case of progressive cancer. According to the result where IL-12 producing abilities with a lapse of time in the administration of ILC and AHCC are compared, the data are more than normal ones on the 7th day which corresponds to early cancer-bearing stage while, on the 10th to 14th days, IL-12 concentration lowers and, on the 14th day which corresponds to medium to later cancer-bearing stage in the case of untreated cancer-bearing mice (B10), the concentration becomes lower than the normal value. On the other hand, in the mice administered with AHCC (1 g/kg/day), the IL-12 productivity becomes highest on the 7th day from the transplantation of tumor (LLC), significantly lowers on the 10th day and significantly decreases on the 14th day. ILX comprises mycelium components where Cortinellus shiitake, Schizophyllum alneum and Merllius lacrymans are compounded in a ratio of 2:2:1 and they are rationally compounded on the basis of the sensitivity of patients to processed mycelia of various mushrooms where immune response of a Th1 system is mainly functioning. When this ILX (1 g/kg/day) is administered, not only on the 7th day but also on the 10th day or even on the 14th day, the IL-12 productivity is maintained in the above cancer-bearing mice.

In mice and human being suffering from progressive cancer and terminal cancer or where immune response of a Th2 system is mainly functioning, PSK which is able to induce IL-12 is administered in a dose of, for example, about 3 g either daily or every other day even in organism in a state of such an immune system. ILY according to the present invention functions as a substitute for PSK. Based upon that, various multi-vitamins of natural type and ursodesoxycholic acid (300 mg or 600 mg/day) and activated vitamin D₃ are jointly administered. If necessary, SPG, OK432 and BCG live vaccine are added as well.

With regard to the NKT cell which is the third main point, saccharide having an α1→3 stereostructure which is a formulation for stimulating the NKR-P1 is applied. Among the cases where the therapy by the above two main points are applied, there were noted the cases of Complete Response (Complete Remission:CR) or Partial Response (PR) in spite of no production of IL-12 in the same ratio as in the cases where IL-12 was produced and the therapy was effective. Now, in order to check whether NKT cells are activated by this novel immunotherapy, investigations were carried out for T cell receptor Vα24Vβ11 of NKT cells and NK cell receptor NKR-P1 (CD 3×CD 161). Incidentally, activation of NKT cells was tested by the ratio (percentage) of CD3⁺CD161⁺ cells (the NKT cells) when total lymphocytes were defined 100%. When the ratio of the NKT cells (CD3⁺CD161⁺) was preferably not less than 10% or, particularly preferably, not less than 16%, it was concluded that NKT cells were activated. As a result, NKR-P1 receptor activated by saccharide having an α1→3 stereostructure such as nigerosaccharide or oligosaccharide showed a positive correlation with the production amount of IL-12 or INFγ while Vα24Vβ11 activated by glycolipid showed a negative correlation therewith. Further, in the cases of CR and PR, there was shown a high correlation with NKR-P1. Thus, in the cases where NITC showed effectiveness, there was suggested a possibility that CTL (cytotoxic T lymphocyte) and another NKT cell act as effector cells.

Accordingly, the above-mentioned composition or composition for oral administration containing a mycelium fraction of Ganoderma boninense according to the present invention which is characterized in jointly using with a composition where the above-mentioned saccharide having an α1→3 stereostructure in which effects of NKT cell activation and IL-12 production induction are able to be achieved at the same time as a main ingredient is further useful for the therapy of cancer and that is able to be an embodiment of the present invention.

Furthermore, an embodiment of the present invention is able to be a supplementary food preparation for oral administration containing the above-mentioned mycelium fraction of Ganoderma boninense according to the present invention which is characterized in jointly using with a composition where the above-mentioned saccharide having an α1→3 stereostructure as a main ingredient.

As mentioned hereinabove, the present invention clarified the relation between a composition where a mycelium fraction of a product obtained by incubation of mycelia of Ganoderma boninense as a main ingredient and ability of induction of the production of IL-12 by the difference in immune response mainly functioning during the progressive stage of cancer or in organism and also shows that the said composition is effective to patients suffering from cancer in a stage of progressive cancer or terminal cancer or in a state where immune response of a Th2 system is mainly functioning and, therefore, when such a thing is carried on a commercial medium, that becomes a discriminating means for the value of the said product. Accordingly, a commercial medium on which such information is carried is highly useful. The above-mentioned commercial medium means printed matters such as pamphlet, booklet or publication, magnetic recording medium such as floppy disk (FD), MO or CD-ROM, information transmitting medium to broad areas such as internet, and the like. Moreover, when such information is commercially utilized, that becomes a discriminating means for the value of the said product and, therefore, the commercial method utilizing the information is of very high utility.

### EXAMPLES

The present invention will now be more specifically illustrated by way of the following Examples although the present invention is not limited thereto but is able to be variously applied within such an extent that they are not beyond the technical idea of the present invention.

### Example 1

Mycelia of Ganoderma boninense were subjected to a liquid culture, the mycelia were centrifuged (at 5,000˜10,000 rpm for 10˜30 minutes) from the resulting culture liquid of mycelia to give a solid fraction and the said fraction was washed with water and 95% ethanol and dried for one night at 85° C. The dried powdery sample of the resulting mycelium fraction was made into a suspension in distilled water and that was used as a sample (hereinafter, it may be referred to as SM).

### Experimental Example 1

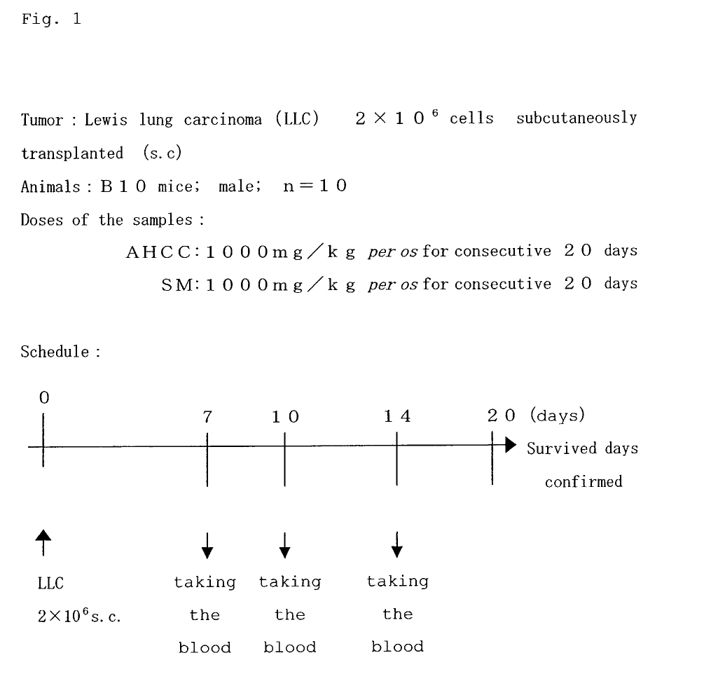

With regard to an inducing ability for the production of IL-12 by the sample (SM) prepared in Example 1 in cancer-bearing organism, an investigation was carried out using cancer-bearing mice to which Lewis lung carcinoma (LLC) was subcutaneously transplanted according to the experimental protocol shown in FIG. 1. Further, AHCC (K. K. Amino Up) was used as a positive control where the inducing ability for the production of IL-12 was known.

As to the test animals, C57/BL10SLC mice (B10 mice) purchased from K. K. SLC were used. In addition to an SM-administered group and an AHCC-administered group of the cancer-bearing mice, the test was conducted in four groups as a whole including an untreated cancer-bearing group and a normal mice group of the same strain as comparative control groups.

As to the tumor to be transplanted, Lewis lung carcinoma was used. Lewis lung carcinoma (2×10⁶ cells) was subcutaneously transplanted to the armpit of B10 mice (male) to prepare cancer-bearing mice.

Dose of SM or AHCC was 1,000 mg/kg (0.1 ml/10 g mouse body weight) per day and that was orally administered for consecutive 20 days.

The day on which Lewis lung carcinoma was subcutaneously transplanted to mice was defined as the starting date for administration of SM or AHCC and blood was collected after 7, 10 and 14 days. Serum which was prepared by a conventional method from the collected blood was subjected to a measurement for IL-12 which was an anti-tumor cytokine and for IAP which was an immunosuppressive substance. Measurement of IL-12 was carried out according to the Directions for Use using a Total Mouse Interleukin-12 Kit (manufactured by Genzyme). Measurement of IAP was carried out according to the Directions for Use using a Mouse IPA Plate (manufactured by K. K. Saikin Kagaku Kenkyusho; sold by Sanko Junyaku K. K.). After 19 days from the starting date of administration of SM, size {[(long diameter)×(short diameter)²]/2 mm³} of the tumor was measured and the tumor-suppressive rate of SM was calculated. Further, even after administration of SM for 20 days, breeding of the cancer-bearing mice to be tested was continued and survival days (life-prolonging effect) were observed.

The result is shown in Tables 1 to 3.

1) Ups and Downs of IL-12 (pg/ml) in Serum (Table 1)

In untreated cancer-bearing mice, IL-12 concentration in serum became higher than the normal value on the 7th day corresponding to the early stage of cancer but, on the 10th to 14 days, the IL-12 concentration lowered and, on the 14th day corresponding to the medium and later stages of cancer, it became lower than the normal value. How to make the concentration of IL-12 high is the problem which is to be solved by the invention.

As shown in Table 1, the production of anti-tumor cytokine IL-12 with a lapse of time in the cancer-bearing mice to which SM (the mycelium fraction of Ganoderma boninense) was administered on the 7th day after the administration was higher than the normal mice but lower than the untreated cancer-bearing mice. On the 10th day from the administration, IL-12 producing ability of the mycelium fraction of Ganoderma boninense was noted and, as a result, IL-12 suddenly increased and, even on the 14th day from the administration, a high productivity was still maintained. The intensity of the productivity was about 1.7-fold of that of the untreated cancer-bearing mice. On the other hand, in the case of AHCC, productivity of IL-12 became the highest on the 7th day from the administration and its amount was nearly the same as that on the 10th day from the administration of the mycelium fraction of Ganoderma boninense. However, on the 10th day after administration of AHCC, its IL-12 productivity lowered and was only 1.2-fold of that of the untreated cancer-bearing mice. Thus, in the case of the mycelium fraction of Ganoderma boninense, the productivity in early stage (7th day) was low as compared with AHCC but, on the 10th day, it became the highest and, after that, the high productivity continued for a long period even after the 14th day. It was found that, with regard to the productivity of anti-tumor cytokine IL-12, the mycelium fraction of Ganoderma boninense was far better than AHCC (a positive control substance) in both terms of the amount and duration of the effect. Therefore, it was ascertained that the mycelium fraction of Ganoderma boninense was able to be expected to have an inducing ability for the production of IL-12 characteristically to the terminal cancer of the progressed stage.

TABLE 1
Amount of IL-12 in Serum (pg/ml)

| | Day | | |
|---|---|---|---|
| Group | 7th | 10th | 14th |
| Normal | 1382.7 ± 313.4 | 1461.5 ± 515.1 | 1436.3 ± 119.4 |
| Cancer-bearing | 1896.3 ± 569.1 | 1416.2 ± 621.3 | 1141.9 ± 306.6 |
| (untreated) | | | |
| Cancer-bearing + | 1580.8 ± 223.9 | 2182.3 ± 1160.2 | 1891.7 ± 944.8 |
| SM | | | |
| Cancer-bearing + | 2140.8 ± 460.4 | 1635.7 ± 866.0 | 1026.2 ± 365.5 |
| AHCC | | | |

2) Ups and Downs of IAP (mg/ml) in Serum (Table 2)

In the untreated cancer-bearing mice, the concentration of IAP which was an anti-tumor and immunosuppressive protein in serum was more than the normal value already on the 7th day which was the early stage of the cancer and was significantly high on the 10th and the 14th days corresponding to medium and later stages of cancer as compared with the normal value. Thus, as the cancer progresses, the concentration of IAP becomes higher.

On the 7th day from the administration, the mycelium fraction of Ganoderma boninense showed an IAP-suppressive action of about 7% as compared with the untreated cancer-bearing mice and, on the 10th day, it showed a suppressive action of as high as about 60%. On the 14th day from the administration, there was shown a suppressive action of about 20% and the IAP-suppressive effect of the mycelium fraction of Ganoderma boninense continued for a long period.

On the contrary, in the AHCC-administered group which was a positive control, no suppressive action was noted on the 7th days from the administration and, on the 10th day, the suppressive rate was about 30% which was only about one-half of that of the mycelium fraction of Ganoderma boninense. On the 14th day from the administration of AHCC, there was shown a suppressive effect of about 10% and, even at that stage, the suppressive rate was only about one-half of the mycelium fraction of Ganoderma boninense. Thus, the mycelium fraction of Ganoderma boninense suppressed the IAP of the cancer-bearing mice for a long period during its administration and its effect was about two-fold of that of AHCC.

TABLE 2
IAP (μg/ml) in Serum

| | Day | | |
|---|---|---|---|
| Group | 7th | 10th | 14th |
| Normal | 236.7 ± 11.6 | 227.0 ± 46.9 | 218.0 ± 77.0 |
| Cancer-bearing | 364.0 ± 56.0 | 1476.0 ± 653.9 | 1826.0 ± 155.0 |
| (untreated) | | | |
| Cancer-bearing + | 337.0 ± 29.1 | 569.0 ± 591.0 | 1474.0 ± 204.0 |
| SM | | | |
| Cancer-bearing + | 391.0 ± 128.7 | 1045.0 ± 657.2 | 1646.0 ± 426.6 |
| AHCC | | | |

3) Survival Rate (%)

A prolonging effect for living days of cancer-bearing mice by administration of a mycelium fraction of Ganoderma boninense was about 150% of that of untreated cancer-bearing mice. Onthecontrary, in the mice being administered with AHCC which was a positive control, no life-prolonging effect was noted at all. In the survival rate of cancer-bearing mice, it was also found that the mycelium fraction of Ganoderma boninense was better than AHCC.

4) Size of Tumor (mm³) (Table 3)

Size of the tumor for each of the cancer-bearing mice was calculated by the following expression.

Size of tumor (mm³)=(long diameter of tumor (mm))×[(short diameter of tumor (mm)]²/2

Suppressive rate for the tumor size of cancer-bearing mice by the mycelium fraction of Ganoderma boninense was calculated by the following expression.

Tumor suppressive rate (%)={1−[(tumor of SM-treated cancer-bearing mice (cancer-bearing+SM))/(tumor of untreated cancer-bearing mice (cancer-bearing+(untreated))]}×100

Although suppression of tumor was not clear on the 7th day after administration of the mycelium fraction of Ganoderma boninense, the tumor suppressing rate showed the highest value of 56. 5% on the 10th day after the administration. The mycelium fraction of Ganoderma boninense showed the tumor suppressive rates of 44.7% and 29.3% on the 14th day and the 19th day, respectively, from the administration. As such, the suppressive rate of the mycelium fraction of Ganoderma boninense for the size of tumor was very high.

TABLE 3
Tumor Suppressive Rate (%) to Tumor Size (mm³)

| | Day | | | |
|---|---|---|---|---|
| Group | 7th | 10th | 14th | 19th |
| Cancer-bearing | 406.9 ± | 2448.2 ± | 4303.1 ± | 661.0 ± 3450.4 |
| (untreated) | 127.6 | 872.0 | 1295.0 | |
| Cancer-bearing + SM | 427.4 ± | 1064.0 ± | 2379.0 ± | 467.5 ± 292.7 |
| | 297.0 | 752.3 | 1333.0 | |
| Suppressive Rate (%) | −5.0 | 56.5 | 44.7 | 29.3 |

### Experimental Example 2

With regard to the inducing ability of the sample (SM) prepared in Example 1 for the production of IL-12 in cancer-bearing organism, an investigation as same as in Experimental Example 1 was carried out again using cancer-bearing mice to which Lewis lung carcinoma (LLC) was subcutaneously transplanted. ILX was used as a positive control where the inducing ability for the production of IL-12 was known already.

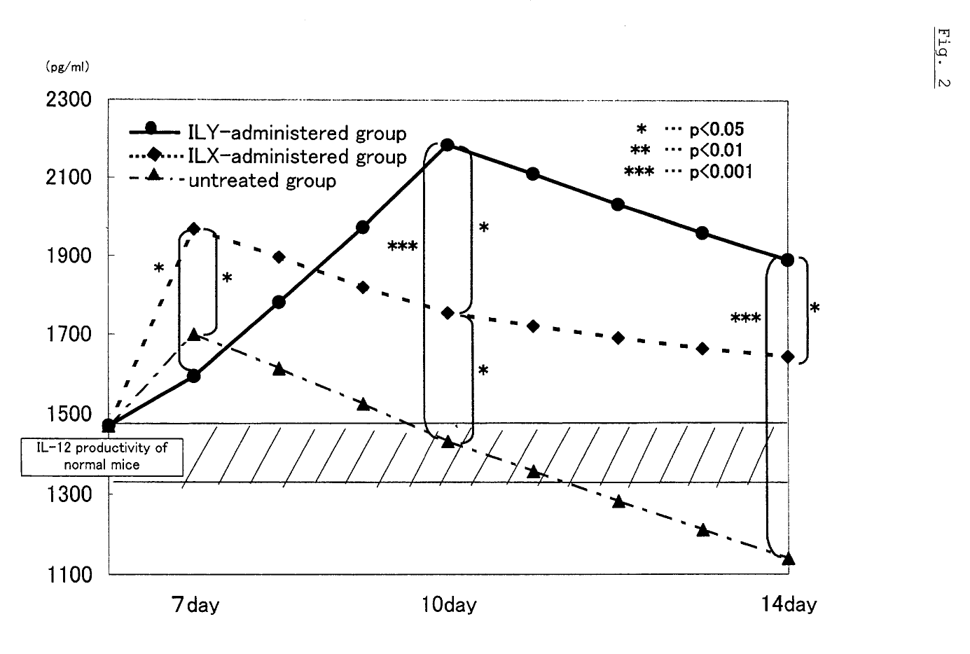

The result is shown in FIG. 2. In FIG. 2, SM is expressed as ILY. In the untreated cancer-bearing mice (expressed by ▴ in FIG. 2), the concentration of IL-12 in serum was higher than the normal value on the 7th day corresponding to early stage of cancer bearing while, on the 10th to 14th days, the IL-12 concentration lowered and, on the 14th day corresponding to middle and latter stages of cancer bearing, it became lower than the normal value. Production of anti-tumor cytokine IL-12 with a lapse of time in the cancer-bearing mice (expressed by ● in FIG. 2) to which the mycelium fraction of Ganoderma boninense was administered was lower than that of the untreated cancer-bearing mice on the 7th day after the administration while, on the 10th day after the administration, the IL-12 producing ability of the mycelium fraction of Ganoderma boninense was noted whereby IL-12 significantly increased and, even on the 14th day after the administration, a high productivity was still maintained. Intensity of the productivity was about 1.7-fold as compared with the untreated cancer-bearing mice and the significance was still noted even on the 10th day and the 14th day after the administration. On the other hand, in the cancer-bearing mice to which ILX was administered (expressed by ♦ in FIG. 2), productivity of IL-12 became the highest on the 7th day after the administration and, although the IL-12 productivity lowered on the 10th day being lower than the cancer-bearing mice administered with ILY, production of IL-12 was still noted even on the 14th day. Thus, the mycelium fraction of Ganoderma boninense showed lower productivity in the early stage (7th day) as compared with ILX but became the highest on the 10th day and, after that, such a high productivity continued for a long period even on the 14th day and thereafter. It was therefore ascertained that the mycelium fraction of Ganoderma boninense was able to be expected for showing its inducing ability for the production of IL-12 characteristically to the terminal cancer in a progressed stage.

### CLINICAL EXAMPLES

As hereunder, there will be mentioned Clinical Examples where dried powder sample of the mycelium fraction of Ganoderma boninense prepared in Example 1 was used and administered to patients suffering from cancer. The test items such as immune parameters and tumor markers measured in each clinical example were measured by the corresponding known means. The said sample is expressed as ILY in the table. Figures given in the lower line for each test item in the table show the normal value for each item. Effectiveness of the therapy used is expressed in the table as Completely Response (Completely Remission:CR), Partially Cured (PC), No Change (no progress of cancer) (NC) or invalid or Progressive Disease (PD) in accordance with the Standard for judgment of the efficacy of anti-cancer agent under GCP of the Japan Ministry of Health and Welfare.

### Clinical Example 1

### Table 4

Male of 62 Years Age; Cancer of Right Ureter and Prostatic Cancer

The case is a male suffering from cancer of right ureter and had an operation of excision of right kidney on Feb. 1, 1994. Prostatic cancer was also found at that time and he has been having a hormone therapy of Leuplin once a month.

In the first medical examination on Apr. 6, 1998, PA (PSA) was as high as 12 μg/ml (not more than 4.0) and a novel immunotherapy (NITC) was started. TNFα, IFNγ and IL-12 were well produced. Although a slight increase was noted during 12 months until Jun. 26, 1999, the result was still NC. However, γ-seminoprotein which was a tumor marker (normal value: not more than 4.0 ng/ml) showed an abnormal value of 4.40 ng/ml for the first time and, after that, PA (PSA) and γ-seminoprotein increased.

Starting from Oct. 27, 2000, additional administration of 3.0 g ILY/every other day was carried out to the novel immunotherapy containing usual ILX. As a result, NKT cell numbers increased to about 16%, NKT perforin value became not less than 4.3% as well, production amount of IL-12 also increased and both PA (PSA) and γ-seminoprotein became normal as well. It is likely that such a result is due to an induction of synergism by ILY.

### Clinical Example 2

### Table 5

Male of 73 Years Age; Adenocarcinoma of Right Lung and Metastasis to Lacunar Lymph Node of Left Clavicle

The case is a male of 73 years age suffering from adenocarcinoma of right lung and is in a stage 1V where excision is not possible.

NITC was started from Jun. 3, 2000. After that, with regard to tumor markers, all of CEA, TPA, BCA 225 and sialyl LEX-1 (SLX-1) increased.

After 4 months, 3.0 g ILY were additionally administered every other day. After that, IL-12 was produced, NKT cell numbers were also maintained and there was noted such a lowering tendency that 164 ng/ml (not more than 5.0) of CEA, 98 U/ml (not more than 70) of TPA, 110 U/ml (not more than 160) of BCA and 130 U/ml (not more than 38) of SLX-1 whereby the case was judged to be PR.

### Clinical Example 3

### Table 6

Female of 45 Years Age; Left Breast Cancer and Multiple Metastases to Bones

The case is a female of 45 years age suffering from left breast cancer and multiple metastases to bones.

NITC was started from Jun. 17, 1999. Various tumor markers lowered and became normal (PR) except ICTP. However, from Sep. 16, 2000, CEA and TPA became 5.6 ng/ml (not more than 5.0) and 120 U/ml (not more than 70), respectively and ICTP also showed abnormal value to result in PD.

Administration of 3.0 g of ILY (being divided into three) every other day was started from Oct. 28, 2000. After that, NKT cell numbers and IL-12 significantly increased and various tumor markers lowered. It is likely that such a result showed that immune ability was improved and tumor tended to become small by the additional administration of 3.0 g of ILY every other day.

### Clinical Example 4

### Table 7

Female of 74 Years Age; Right Breast Cancer and Metastasis to Lung and Multiple Metastases to Bones

This is the case suffering from right breast cancer and metastasis to lung and multiple metastases to bones and is the case of terminal cancer being ineffective by anticancer agents and radioactive ray.

Since infiltration of cancer was too much progressive at the starting stage of NITC on Jun. 24, 2000, a joint administration with 3.0 g of ILY every other day was carried out from the initial stage. After starting the therapy for the terminal cancer, tumor markers were significantly improved and, on Mar. 3, 2000, all tumor markers became normal and abnormal observation disappeared in a CT bone scintigram as well. That is likely to be a result where synergism by the effect of NITC with ILY was induced.

### Clinical Example 5

### Table 8

Female of 64 Years Age; Stomach (Scirrhous) Cancer

This is the case of female of 64 years age suffering from stomach (scirrhous) cancer where excision is not possible.

NITC was started from Feb. 16, 1998. Tumor markers were improved and the case was judged to be PR until Aug. 7, 1999.

After that, tumor markers increased and the case was judged to be PD. From Oct. 27, 2000, administration of 3.0 g of ILY every other day was started. After that, as the productivity of IL-12 increased, tumor markers decreased whereby the case was judged to be PR. Such an effect is also likely to be due to synergism with ILY.

### Clinical Example 6

### Table 9

Female of 47 Years Age; Right Breast Cancer and Recurrence in Cervical Lymph Node

This is the case of recurrence of cervical lymph node in breast cancer.

NITC was started whereupon tumor markers significantly decreased being judged to be CR. After one month however, recurred enlargement of cervical lymph node appeared.

ILY (3.0 g) every other day was added from Oct. 28, 2000. From 2 months after the administration, an increase in the tumor markers ceased and, during about 3 months, the state of NC was maintained. The action of induction of NC as such was presumed to be due to ILY.

### Clinical Example 7

### Table 10

Male of 60 Years Age; Rectum Cancer and Recurrence in Abdominal Lymph Node

This is the case of male of 60 years age where metastasis to abdominal lymph node was noted after the operation of rectum cancer.

During 15 months after starting of NITC, tumor markers were within a normal level but, from Nov. 13, 1999, CEA became an abnormal level of 6.0 ng/ml (not more than 5.0) and increased thereafter.

From Dec. 9, 2000, 3.0 g of ILY were additionally administered every other day. After that, tumor markers did not increase and potentiation of production of IL-12 and NKT cells was resulted. Such a fact was presumed to be the result of administration of 3.0 g of ILY every other day.

### Clinical Example 8

### Table 11

Female of 46 Years Age; Thymoma and Metastasis to Liver and Lung

This is a case of female of 46 years age suffering from thymoma and metastasis to lung and liver is noted.

Although NITC was carried out from Sep. 8, 2000, tumor markers increased and the case was judged to be PD.

From Dec. 22, 2000, 3.0 g of ILY were additionally administered every other day. After 2 months, IL-12 production was potentiated and an increase in tumor markers ceased as well. Such a result was likely to be due to potentiation of immunity by ILY.

TABLE 4
Clinical Example 1

| | | | | CD3+ | | | | | | | | | γ - | |
|---|---|---|---|---|---|---|---|---|---|---|---|---|---|---|
| | Diag- | | | CD161 | Ratio of | | | | | | PAP | PA | Semino- | |
| | nosed | | CD3+ | + | TH1/TH2 | TNF α | IFN γ | IL-12 | | | (EIA) | (RIA) | protein | 1CTP |
| | Period | Effi- | CD161+ | Perforin | (CD4) | (1000 | (10 | (7.8 | IL-10 | VEGF | (3.0 | (4.0 | (4.0 | (4.5 |
| | (months) | cacy | (16%) | (4.3%) | (≧7) | pg/ml) | IU/ml) | pg/ml) | (pg/ml) | (pg/ml) | ng/ml) | ng/ml) | ng/ml) | ng/ml) |
| | 11 | NC | | | | | | | | | 1.3 | 12 | 2.4 | 4.5 |
| | 16 | NC | | | | 1760 | 45.2 | 33.4 | | | 1.5 | 13 | 3.5 | 4.1 |
| | 20 | NC | | | | 4700 | 41.1 | 39.6 | | | 1.8 | 15 | 3.7 | 4.4 |
| | 23 | NC | | | 5.6 | 2410 | 35.0 | 50.3 | 951 | | 1.8 | 15 | 3.8 | 4.3 |
| | 26 | NC | | | 5.5 | 410 | 17.8 | 7.8> | 904 | | 1.7 | 15 | 4.4 | 4.1 |
| | 29 | PD | | | 11.7 | 350 | 9.7 | 7.8> | 438 | | 1.4 | 16 | 4.1 | 3.7 |
| | 31 | PD | 5.0 | | 9.8 | 310 | 6.1 | 7.8> | 686 | | 1.3 | 17 | 5.0 | 4.8 |
| | 32 | NC | | | 14.3 | 800 | 20.4 | 7.8> | 623 | | 1.7 | 22 | 6.6 | 3.4 |
| | 33 | NC | 6.1 | | 10.0 | 500 | 24.7 | 7.8> | 59 | | | 26 | 7.2 | 4.8 |
| | 35 | NC | | | 11.0 | 1050 | 19.6 | 7.8> | 403 | | 2 | 22 | 6.0 | 3.8 |
| | 38 | NC | 9.3 | | 12.2 | 1580 | 40.5 | 16.5 | 895 | | 2.1 | 23 | 8.9 | 3.6 |
| | 41 | NC | 6.4 | | 8.9 | 2500 | 61.0 | 19.6 | 783 | | | 22 | 8.5 | 4.2 |
| ILY | 43 | NC | | | | | | | | | 1.8 | 26 | 7.3 | 4.3 |
| started | | | | | | | | | | | | | | |
| | 44 | PR | 11.5 | 3.9 | 9.7 | 4630 | 61.0 | 21.2 | 1020 | 296 | 1.0 | 11 | 2.6 | 3.6 |
| | 44.5 | CR | | | | | | | | | 0.6 | 2.2 | 0.4 | 4 |
| | 45 | CR | | | | | | | | | | | 0.6 | 3.8 |
| | 47 | CR | 15.7 | 5.3 | 9.2 | | | | | 278 | 0.5 | 0.2> | 0.3> | 4.8 |

TABLE 5
Clinical Example 2

| | | | | CD3+ | Ratio of | | | | | | | | | | |
|---|---|---|---|---|---|---|---|---|---|---|---|---|---|---|---|
| | | | CD3+ | CD161 | TH1/ | | | | | | | | | sialyl | |
| | Diagnosed | | CD161 | + | TH2 | TNF α | IFN γ | IL-12 | | | CEA | TPA | BCA225 | LEX-1 | 1CTP |
| | Period | Effi- | + | Perforin | (CD4) | (1000 | (10 | (7.8 | IL-10 | VEGF | (5.0 | (70 | (160 | (38 | (4.5 |
| | (months) | cacy | (16%) | (4.3%) | (≧7) | pg/ml) | IU/ml) | pg/ml) | (pg/ml) | (pg/ml) | ng/ml) | U/l) | U/ml) | U/ml | ng/ml) |
| | 0 | PD | 22.1 | | 13.2 | 980 | 6.8 | 7.8> | 897 | | 44 | 36 | 160 | 73 | 5.9 |
| | 1 | PD | 21.9 | | 12.7 | 2790 | 20.8 | 7.8> | 1200 | | 72 | | | 90 | 5.7 |
| | 3 | PD | | | | | | | | | 140 | 86 | 170 | 100 | 6.3 |
| | 4 | PD | 21.2 | | 10.4 | 530 | 3.9 | 7.8> | 403 | | 166 | 110 | 150 | 130 | 5.9 |
| ILY | 5 | PD | | | | | | | | | 190 | 99 | 150 | 140 | 6 |
| start- | | | | | | | | | | | | | | | |
| ed | | | | | | | | | | | | | | | |
| | 6 | PD | 26.3 | 16 | 10.4 | 1310 | 1.6 | 7.8> | 668 | 228 | 191 | 140 | 130 | 170 | 5.2 |
| | 7 | NC | 21.1 | 10.9 | 7 | 1170 | 18.1 | 16.8 | 311 | 538 | 176 | 100 | 140 | 190 | 8.1 |
| | 8 | PR | 14.7 | 10.1 | 32.2 | 2560 | 24.2 | 26.1 | 167 | 241 | 164 | 98 | 110 | 130 | 7.9 |

TABLE 6
Clinical Example 3

| | | | | | Ratio | | | | | | | | | |
|---|---|---|---|---|---|---|---|---|---|---|---|---|---|---|
| | | | | CD3+ | of | | | | | | | | | |
| | Diagnosed | | CD3+ | CD161+ | TH1/TH2 | TNF α | IFN γ | IL-12 | | | CEA | TPA | CA15-3 | 1CTP |
| | Period | Effi- | CD161+ | Perforin | (CD4) | (1000 | (10 | (7.8 | IL-10 | VEGF | (5.0 | (70 | (30 | (4.5 |
| | (months) | cacy | (16%) | (4.3%) | (≧7) | pg/ml) | IU/ml) | pg/ml) | (pg/ml) | (pg/ml) | ng/ml) | U/l) | U/ml) | ng/ml) |
| | 2 | PR | | | 12.1 | 1170 | 9.8 | 7.8> | 922 | | 8.0 | | | |
| | 3 | PR | | | 10.5 | 2200 | 15.1 | 9.4 | 733 | | 13.8 | | 42 | |
| | 6 | PR | | | 13.8 | 300 | 1.8 | 7.8> | 352 | | 17.8 | | 31 | 27.0 |
| | 10 | PR | | | 10.6 | 550 | 5.1 | 7.8> | 484 | | 1.4 | | 12 | 11.3 |
| | 12 | PR | 19.0 | | 10.0 | 390 | 7.7 | 7.8> | 749 | | 1.4 | | 17 | 8.6 |
| | 14 | PR | 16.5 | | 8.1 | 460 | 4.6 | 7.8> | 939 | | 1.8 | 58 | 19 | 8.1 |
| ILY | 17 | PD | | | | | | | | | 8.4 | 220 | 49 | 8.9 |
| started | | | | | | | | | | | | | | |
| | 18 | PD | 12.9 | 3.9 | 11.6 | 680 | 4.6 | 7.8> | 691 | 447 | 8.1 | 190 | 64 | 13.5 |
| | 19 | PR | | | | | | | | 429 | 6.9 | 220 | 50 | 12.7 |
| | 20 | PR | | | | | | | | 396 | 4.9 | 210 | 54 | 12.6 |
| | 21 | PR | 18.9 | 4.9 | 8.6 | 4600 | 69.5 | 26.1 | 596 | | 4.2 | 120 | | 8.4 |

TABLE 7
Clinical Example 4

| | | | | CD3+ | Ratio | | | | |
|---|---|---|---|---|---|---|---|---|---|
| | | | CD3+ | CD161 | of | | | | |
| | Diagnosed | | CD161 | + | TH1/TH2 | TNF α | IFN γ | IL-12 | |
| | Period | Effi- | + | Perforin | (CD4) | (1000 | (10 | (7.8 | IL-10 |
| | (months) | cacy | (16%) | (4.3%) | (≧7) | pg/ml) | IU/ml) | pg/ml) | (pg/ml) |
| ILY | 0 | PR | 14.6 | | 5.1 | 400 | 0.9 | 7.8> | 161 |
| started | | | | | | | | | |
| | 1 | PR | | | | | | | |
| | 2 | PR | 20.8 | | 1.8 | 610 | 0.7 | 7.8> | 227 |
| | 3 | PR | | | | | | | |
| | 3.5 | PR | | | | | | | |
| | 4 | PR | 12.3 | 2.8 | 7.3 | 210 | 24.0 | 14 | 392 |
| | 5 | PR | | | | | | | |
| | 6 | CR | 17.4 | 4.1 | 2.5 | 4310 | 34.2 | 23 | 334 |
| | 8 | CR | 12.7 | 6.1 | 8.1 | 1690 | 25.6 | 24 | 238 |

| | | | | | NCC- | | | | |
|---|---|---|---|---|---|---|---|---|---|
| | | | | | ST- | | | Sialyl | |
| | Diagnosed | | CEA | TPA | 439 | CA15-3 | BCA225 | LEX-1 | 1CTP |
| | period | VEGF | (5.0 | (70 | (7.0 | (30 | (160 | (38 | (4.5 |
| | (months) | (pg/ml) | ng/ml) | U/l) | U/ml) | U/ml) | U/ml) | U/ml) | ng/ml) |
| ILY | 0 | | 1.2 | 1500 | 110.0 | 170.0 | 270 | 44 | 8.9 |
| started | | | | | | | | | |
| | 1 | | 1> | 440 | 86.0 | 130.0 | 380 | 45 | 10.5 |
| | 2 | | | 110 | 75.0 | 110.0 | 310 | | 11.5 |
| | 3 | | | 58 | 40.0 | 50.0 | 220 | 31 | 10.2 |
| | 3.5 | | | 54 | 24.0 | 39.0 | 170 | 30 | 7.4 |
| | 4 | 202 | | 18 | 5.5 | 19.0 | 73 | 22 | 6.9 |
| | 5 | 226 | 1.8 | | 1.0> | 11.0 | 51 | 23 | 5.0 |
| | 6 | | 1.7 | 29 | 1.8 | 7.5 | 61 | 29 | 4.9 |
| | 8 | | | 20 | 1.6 | 6.0 | 24 | 22 | 4.1 |

TABLE 8
Clinical Example 5

| | | | | CD3+ | | | | | |
|---|---|---|---|---|---|---|---|---|---|
| | | | CD3+ | CD161 | Ratio of | | | | |
| | Diagnosed | | CD161 | + | TH1/TH2 | TNF α | IFN γ | IL-12 | |
| | period | Effi- | + | Perforin | (CD4) | (1000 | (10 | (7.8 | IL-10 |
| | (months) | cacy | (16%) | (4.3%) | (≧7) | pg/ml) | IU/ml) | pg/ml) | (pg/ml) |
| | 2 | PR | | | | | | | |
| | 3 | PR | | | | 2010 | 3.0 | 7.8> | |
| | 6 | PR | | | | 1220 | 28.3 | 8.1 | |
| | 10 | PR | | | | 1360 | 15.9 | 11.2 | |
| | 13 | PR | | | 5.7 | 2680 | 43.7 | 11.6 | |
| | 15 | PR | | | 3.2 | 3320 | 46.9 | 58.9 | 692 |
| | 20 | PD | | | 4.3 | 480 | 4.4 | 7.8> | 478 |
| | 23 | PD | | | 5.0 | 4800 | 108.0 | 26.1 | 1480 |
| | 24 | PD | 12.9 | | 2.9 | 2850 | 52.3 | 18.1 | 665 |
| | 26 | PD | | | 3.9 | 290 | 6.8 | 7.8> | 622 |
| | 29 | PD | 17.5 | | 3.8 | 680 | 6.2 | 7.8> | 844 |
| | 31 | PD | 12.9 | | 2.4 | 440 | 10.0 | 7.8> | 667 |
| ILY | 33 | PD | | | | | | | |
| started | | | | | | | | | |
| | 34 | PD | 15.2 | 3.1 | 3.9 | 2250 | 33.0 | 7.8> | 1390 |
| | 35 | PR | | | | | | | |
| | 36 | PR | | | | | | | |
| | 37 | PR | 13.8 | 2.3 | 3.0 | 1050 | 46.2 | 26.7 | 601 |

| | | | | | | STN | | |
|---|---|---|---|---|---|---|---|---|
| | Diagnosed | | CEA | CA19-9 | CA72-4 | antigen | IAP | 1CTP |
| | period | VEGF | (5.0 | (7.0 | (4.0 | (45 | (500 | (4.5 |
| | (months) | (pg/ml) | ng/ml) | U/ml) | U/ml) | U/ml) | μg/ml) | ng/ml) |
| | 2 | | 7.4 | 220 | 3.6 | 38 | 158 | |
| | 3 | | 4.4 | 240 | 3.0 | 37 | 176 | |
| | 6 | | 6.6 | 66 | | 37 | 67 | 8.0 |
| | 10 | | 7.0 | 58 | | 41 | 54 | 7.8 |
| | 13 | | 9.6 | 66 | | | 60 | 5.2 |
| | 15 | | 8.8 | 85 | 14.0 | | | 6.2 |
| | 20 | | 5.6 | 180 | 16.0 | 35 | | 6.8 |
| | 23 | | 5.2 | 450 | 3.0 | 30 | 74 | 6.3 |
| | 24 | | 7.2 | 510 | 3.0 | 29 | | 4.8 |
| | 26 | | 6.2 | 670 | 6.1 | 37 | | 6.1 |
| | 29 | | 5.6 | 1100 | | 41 | 41 | 5.0 |
| | 31 | | 4.0 | 1800 | | | | 5.1 |
| ILY | 33 | | 5.4 | 2000 | 12.0 | | | 5.6 |
| started | | | | | | | | |
| | 34 | 227 | 4.6 | 2500 | 18.0 | | | 5.1 |
| | 35 | 236 | 5.5 | 2200 | 24.0 | 47 | | 4.9 |
| | 36 | 232 | 5.5 | 1600 | 12.0 | 51 | | 4.8 |
| | 37 | 249 | 4.7 | 1600 | 6.2 | 42 | 215 | 4.1 |

TABLE 9
Clinical Example 6

| | | | | CD3+ | Ratio of | | | | | | | | | | |
|---|---|---|---|---|---|---|---|---|---|---|---|---|---|---|---|
| | | | CD3+ | CD161 | TH1/ | | | | | | | NCC- | | Sialyl | |
| | Diagnosed | | CD161 | + | TH2 | TNF α | IFN γ | IL-12 | | | CEA | ST-439 | CA15-3 | LEX-1 | 1CTP |
| | period | Effi- | + | Perforin | (CD4) | (1000 | (10 | (7.8 | IL-10 | VEGF | (5.0 | (7.0 | (30 | (38 | (4.5 |
| | (months) | cacy | (16%) | (4.3%) | (≧7) | pg/ml) | IU/ml) | pg/ml) | (pg/ml) | (pg/ml) | ng/ml) | U/ml) | U/ml) | U/ml) | ng/ml) |
| | 0 | CR | | | | 1500 | 3.8 | 7.8> | | | 3.2 | 56.0 | 19 | | 2.3 |
| | 3 | CR | | | | 1360 | 11.9 | 7.8> | | | 3.6 | 3.6 | 11 | 28 | 3 |
| | 8 | CR | | | 9.7 | 1770 | 11.6 | 16.7 | 291 | | 4 | 1.3 | 12 | 29 | 2.2 |
| | 11 | CR | | | 13.5 | 126 | 2.2 | 7.8> | 264 | | 4.6 | 1.9 | 9.9 | 28 | |
| | 15 | CR | 14.8 | | 13.4 | 460 | 8.4 | 7.8> | 269 | | 4.6 | 6.5 | 11 | 24 | 1.9 |
| | 20 | PD | 18.7 | | 20.5 | 300 | 4.4 | 7.8> | 408 | | 5.2 | 42.0 | 24 | 30 | 1.7 |
| | 23 | PD | 16.8 | | 9.3 | 470 | 7.7 | 7.8> | 610 | | 3.8 | 67.0 | 24 | 25 | 1.9 |
| ILY | 24.5 | PD | | | | | | | | | 4.4 | 88.0 | 31 | 28 | 1.8 |
| start- | | | | | | | | | | | | | | | |
| ed | | | | | | | | | | | | | | | |
| | 25 | PD | | | | | | | | 1150 | | 73.0 | | | 1.9 |
| | 25.5 | PD | | | | | | | | | 3.7 | 100.0 | | | 2.6 |
| | 26 | PD | 16.4 | 2.8 | 11.2 | 2540 | 27.8 | 15.2 | 698 | 1180 | 3.6 | 100.0 | 39 | 35 | 2 |
| | 26.5 | NC | | | | | | | | 980 | | 110.0 | | | 2.1 |
| | 27 | NC | | | | | | | | | | 110.0 | | | 1.7 |
| | 28 | NC | 22.1 | 3.7 | 13.2 | 1060 | 8.4 | 7.8> | 568 | | 3.5 | 130.0 | 36 | 29 | 2.5 |
| | 29 | NC | | | | | | | | 1500 | 3.2 | 140.0 | 31 | 32 | 2 |

TABLE 10
Clinical Example 7

| | | | | CD3+ | Ratio of | | | | | | | | |
|---|---|---|---|---|---|---|---|---|---|---|---|---|---|
| | Diagnosed | | CD3+ | CD161+ | TH1/TH2 | TNF α | IFN γ | IL-12 | | | CEA | CA19-9 | 1CTP |
| | period | Effi- | CD161+ | Perforin | (CD4) | (1000 | (10 | (7.8 | IL-10 | VEGF | (5.0 | (37 | (4.5 |
| | (months) | cacy | (16%) | (4.3%) | (≧7) | pg/ml) | IU/ml) | pg/ml) | (pg/ml) | (pg/ml) | ng/ml) | U/ml) | ng/ml) |
| | NC | 0 | | | | 4540 | 123 | 24.2 | | | 2.0 | 8 | 4.9 |
| | NC | 3 | | | | 2620 | 67.7 | 16.1 | | | 1.8 | 7 | 4.6 |
| | NC | 9 | | | 12.0 | 4740 | 139.0 | 170.0 | 580 | | 2.4 | 9 | 3.3 |
| | NC | 12 | | | 8.7 | 1930 | 91.3 | 129.0 | 150 | | 34 | 9 | 3.4 |
| | NC | 15 | | | 8.2 | 2270 | 54.1 | 23.2 | 538 | | 4.6 | 13 | 3.2 |
| | PD | 18 | 19.9 | | 15.6 | 3000 | 82.9 | 56.6 | 196 | | 7.0 | 22 | 2.7 |
| | PD | 20 | 18.6 | | 9.1 | 3570 | 106.0 | 77.5 | 402 | | 9.0 | 29 | 3.0 |
| | PD | 22 | 16.3 | | 7.0 | 1950 | 55.0 | 28.5 | 435 | | 13.4 | 45 | 2.1 |
| | PD | 26 | 17.6 | | 4.3 | 2050 | 31.1 | 12.7 | 470 | | 22.0 | 70 | 2.7 |
| | PD | 29 | | | | | | | | 338 | 48.0 | 130 | 2.9 |
| ILY | PD | 30 | 10.3 | 2.6 | 8.7 | 3820 | 60.1 | 26.6 | 578 | 331 | 42.5 | 140 | 3.1 |
| start- | | | | | | | | | | | | | |
| ed | | | | | | | | | | | | | |
| | NC | 31 | | | | | | | | 375 | 55.3 | 150 | 2.4 |
| | NC | 32 | | | | | | | | 327 | 56.3 | 160 | 3.0 |
| | NC | 33 | 16.8 | 7.9 | 6.9 | 3860 | 57.6 | 54.5 | 381 | 431 | 60.8 | 160 | 3.0 |

TABLE 11
Clinical Example 8

| | | | | CD3+ | Ratio of | | | | | | | | | | |
|---|---|---|---|---|---|---|---|---|---|---|---|---|---|---|---|
| | | | CD3+ | CD161 | TH1/ | | | | | | | | NSE | | |
| | Diagnosed | | CD161 | + | TH2 | TNF α | IFN γ | IL-12 | | | TPA | GAT | (RIA) | BFP | 1CTP |
| | period | Effi- | + | Perforin | (CD4) | (1000 | (10 | (7.8 | IL-10 | VEGF | (70 | (13.6 | (10 | (75 | (4.5 |
| | (months) | cacy | (16%) | (4.3%) | (≧7) | pg/ml) | IU/ml) | pg/ml) | (pg/ml) | (pg/ml) | U/l) | U/ml) | ng/ml) | ng/ml) | ng/ml) |
| | PD | 0 | 13.3 | | 14.5 | 4030 | 76.4 | 16.7 | 588 | | 71 | 15.6 | 21 | 170 | 7.1 |
| | PD | 1 | 10.7 | | 12 | 1130 | 28.3 | 7.8> | 726 | | 120 | 17.2 | 26 | 190 | 6.7 |
| | PD | 2 | | | | | | | | 710 | 110 | 19.7 | 38 | 200 | 7.7 |
| | PD | 3 | | | | | | | | 1030 | 340 | 29.9 | 53 | 310 | 10.3 |
| ILY | PD | 3.5 | | | | | | | | 1600 | 230 | 45.4 | 75 | 430 | 10.2 |
| start- | | | | | | | | | | | | | | | |
| ed | | | | | | | | | | | | | | | |
| | PD | 4 | 18.6 | 6.1 | 33.9 | 5210 | 3.3 | 7.8> | 542 | 2100 | 260 | 51.6 | 110 | 520 | 15.5 |
| | NC | 5 | 18.2 | 6.7 | 34.1 | 4900 | 24.8 | 14.1 | 242 | 2540 | 160 | 41.3 | 110 | 290 | 13.9 |

### INDUSTRIAL APPLICABILITY

The present invention has provided a mycelium fraction of Ganoderma boninense which is effective in inducing the production of IL-12 and clarified the effective administration stage corresponding to the progressive step of cancer in induction of IL-12 production by the mycelium fraction of Ganoderma boninense. Thus, the composition, the composition by oral administration, the supplementary food preparation for health by oral administration, the commercial medium carrying the information concerning the same and the commercial method using the said commercial medium in accordance with the present invention are able to greatly contribute in the fundamental and clinical studies of cancer and also in the therapy of cancer.

1. A composition which induces the production of interleukin 12 (IL-12) containing a mycelium fraction of the product obtained by incubation of mycelia of Ganoderma boninense as a main ingredient.

2. A composition which induces the production of interleukin 12 (IL-12) containing a mycelium fraction of the product obtained by liquid incubation of mycelia of Ganoderma boninense as a main ingredient.

3. A composition for oral administration which induces the production of interleukin 12 (IL-12) containing a mycelium fraction of the product obtained by incubation of mycelia of Ganoderma boninense as a main ingredient.

4. A composition for oral administration which induces the production of interleukin 12 (IL-12) containing a mycelium fraction of the product obtained by liquid incubation of mycelia of Ganoderma boninense as a main ingredient.

5. A composition for oral administration which induces the production of interleukin 12 (IL-12) containing a mycelium fraction of the product obtained by incubation of mycelia of Ganoderma boninense as a main ingredient which is characterized in that, as the dose achieving the inducing ability for the production of IL-12 in vivo, the main ingredient is administered per os in a dose of 100 mg to 2,000 mg/kg body weight/day.

6. A composition for oral administration which induces the production of interleukin 12 (IL-12) containing a mycelium fraction of the product obtained by liquid incubation of mycelia of Ganoderma boninense as a main ingredient which is characterized in that, as the dose achieving the inducing ability for the production of IL-12 in vivo, the main ingredient is administered per os in a dose of 100 mg to 2,000 mg/kg body weight/day.

7. A composition which induces the production of interleukin 12 (IL-12) containing a mycelium fraction of the product obtained by incubation of mycelia of Ganoderma boninense as a main ingredient which is characterized in that it is useful for the patient suffering from progress cancer or terminal cancer.

8. A composition which induces the production of interleukin 12 (IL-12) containing a mycelium fraction of the product obtained by liquid incubation of mycelia of Ganoderma boninense as a main ingredient which is characterized in that it is useful for the patient suffering from progress cancer or terminal cancer.

9. A composition for oral administration which induces the production of interleukin 12 (IL-12) containing a mycelium fraction of the product obtained by incubation of mycelia of Ganoderma boninense as a main ingredient which is characterized in that it is useful for the patient suffering from progress cancer or terminal cancer.

10. A composition for oral administration which induces the production of interleukin 12 (IL-12) containing a mycelium fraction of the product obtained by liquid incubation of mycelia of Ganoderma boninense as a main ingredient which is characterized in that it is useful for the patient suffering from progress cancer or terminal cancer.

11. A composition for oral administration which induces the production of interleukin 12 (IL-12) containing a mycelium fraction of the product obtained by incubation of mycelia of Ganoderma boninense as a main ingredient and is effective to patients suffering from progressive cancer or terminal cancer which is characterized in that, as the dose achieving the inducing ability for the production of IL-12 in vivo, the main ingredient is administered per os in a dose of 100 mg to 2,000 mg/kg body weight/day.

12. A composition for oral administration which induces the production of interleukin 12 (IL-12) containing a mycelium fraction of the product obtained by liquid incubation of mycelia of Ganoderma boninense as a main ingredient and is effective to patients suffering from progressive cancer or terminal cancer which is characterized in that, as the dose achieving the inducing ability for the production of IL-12 in vivo, the main ingredient is administered per os in a dose of 100 mg to 2,000 mg/kg body weight/day.

13. A composition which induces the production of interleukin 12 (IL-12) containing a mycelium fraction of the product obtained by incubation of mycelia of Ganoderma boninense as a main ingredient which is characterized in that it is useful for patients suffering from cancer where a T helper 2 cell system immune response is mainly functioning.

14. A composition which induces the production of interleukin 12 (IL-12) containing a mycelium fraction of the product obtained by liquid incubation of mycelia of Ganoderma boninense as a main ingredient which is characterized in that it is useful for patients suffering from cancer where a T helper 2 cell system immune response is mainly functioning.

15. A composition for oral administration which induces the production of interleukin 12 (IL-12) containing a mycelium fraction of the product obtained by incubation of mycelia of Ganoderma boninense as a main ingredient which is characterized in that it is useful for patients where a T helper 2 cell system immune response is mainly functioning.

16. A composition for oral administration which induces the production of interleukin 12 (IL-12) containing a mycelium fraction of the product obtained by liquid incubation of mycelia of Ganoderma boninense as a main ingredient which is characterized in that it is useful for patients where a T helper 2 cell system immune response is mainly functioning.

17. A composition for oral administration which induces the production of interleukin 12 (IL-12) containing a mycelium fraction of the product obtained by incubation of mycelia of Ganoderma boninense as a main ingredient and is effective to patients suffering from cancer where a T helper 2 cell system immune response is mainly functioning which is characterized in that, as the dose achieving the inducing ability for the production of IL-12 in vivo, the main ingredient is administered per os in a dose of 100 mg to 2,000 mg/kg body weight/day.

18. A composition for oral administration which induces the production of interleukin 12 (IL-12) containing a mycelium fraction of the product obtained by liquid incubation of mycelia of Ganoderma boninense as a main ingredient and is effective to patients suffering from cancer where a T helper 2 cell system immune response is mainly functioning which is characterized in that, as the dose achieving the inducing ability for the production of IL-12 in vivo, the main ingredient is administered per os in a dose of 100 mg to 2,000 mg/kg body weight/day.

19. The composition according to any of claims 1, 2, 7, 8, 13 and 14, wherein the said composition is used together with a composition in which a saccharide having an α1→3 stereostructure as a main ingredient.

20. The composition for oral administration according to any of claims 3 to 6, 9 to 12 and 15 to 18, wherein the said composition is used together with a composition in which a saccharide having an α1→3 stereostructure as a main ingredient.

21. The composition for oral administration according to any of claims 3 to 6, 9 to 12, 15 to 18 and 20, wherein the said composition is a supplementary food preparation for health by oral administration.

22. A commercial medium carrying the content mentioned in any of the above-mentioned claims 1 to 21.

23. A commercial method utilizing the content mentioned in any of the above-mentioned claims 1 to 22.
